(19) United States
(12) Patent Application Publication
(10) Pub. No.: US 2026/0308082 A1
(43) Pub. Date: Oct. 8, 2026

(54) PROCESS AND PLANT FOR PRODUCING SYNTHETIC PARAFFINIC KEROSENE AS JET FUEL

(71) Applicant: Topsoe A/S, Kgs. Lyngby (DK)

(72) Inventors: Rasmus Yding BROGAARD, Kgs. Lyngby (DK); Niels Christian SCHJØDT, Kgs. Lyngby (DK); Esben TAARNING, Kgs. Lyngby (DK)

(21) Appl. No.: 19/481,169

(22) PCT Filed: Apr. 29, 2024
(86) PCT No.: PCT/EP2024/061726

§ 371 Date: Nov. 3, 2025

(30) Foreign Application Priority Data
May 2, 2023 (DK) PA202300387

(51) Int. Cl. C07C 2/30; B01J 8/00; C10L 1/04

(52) U.S. Cl. CPC C07C 2/30; B01J 8/008; C10L 1/04; B01J 2208/00946; C07C 2523/755; C07C 2531/22; C10L 2200/043

(57) ABSTRACT

Processes for producing hydrocarbon products include converting an ethylene stream in an oligomerization reactor by contacting the ethylene stream with an oligomerization catalyst, the oligomerization catalyst being a homogeneous catalyst comprising: a transition metal compound or complex comprising at least one of Ti, Zr, Cr, Ni, with an alkyl aluminum compound dissolved in a hydrocarbon or etheral solvent; or an alkyl aluminum compound dissolved in a hydrocarbon or etheral solvent; for producing a first stream of C2-C20 linear alfa olefins (LAOs); separating the first stream of C2-C20 linear alfa olefins into a second stream of C2-C6 olefins, and a third stream of C8+ olefins; and recycling at least a portion of the second stream of C2-C6 olefins to the oligomerization reactor. Such processes may further include converting the C8+ olefins to synthetic paraffinic kerosene (SPK). Methods of retrofitting existing hydroprocessing plants are also described.

### FIELD OF THE INVENTION

The present invention relates to a process and plant for producing hydrocarbons in the transportation fuel range, for instance a jet fuel such as kerosene, more specifically synthetic paraffinic kerosene (SPK), from ethylene. The ethylene is for instance provided by catalytic dehydration of ethanol. The ethanol is for instance provided by the fermentation of biomass. The present invention relates also to a method of retrofitting existing hydroprocessing plants.

### BACKGROUND OF THE INVENTION

It is known to produce hydrocarbons in the transportation fuel range, such as fuels for aviation (jet fuel, kerosene) from non-fossil sources. This can be accomplished by various technologies such as Fischer-Tropsch (FT) synthesis from non-fossil synthesis gas, or by converting methanol-to-olefins (MTO) from non-fossil methanol combined with oligomerization and hydrogenation. Another feedstock is ethylene, herein also referred to as C2= or C2-olefin, which can be oligomerized and then hydrogenated and fractionated to synthetic paraffinic kerosene (SPK), the SPK being a mixture of alkanes, mostly iso-alkanes, in the C8-C18 range.

Ethanol can be converted to ethylene and oligomerized in various ways, followed by hydrogenation and fractionation to produce SPK, as for instance disclosed in US 2022/0396741, U.S. Pat. Nos. 11,377,607, 11,078,433 and US 20150299069.

It would be desirable to provide a process for converting ethylene to SPK in at least 75%, such as at least 80%, or at least 85%, or at least 90%overall yield and with a freezing point of −40° C. or below, or even −47° C. or below. The processes according to above references, however, are unable to produce as high yield of SPK with such low freezing point as by the process and plant of the present invention. Further, due to the exothermic nature of the oligomerization reactions and the formation of heavy wax or polymeric material, there is a high risk of fouling with attendant plugging of the oligomerization reactor and associated equipment, such as pipes. This may result in frequent plant shutdown, which is highly undesirable. Furthermore, in existing hydroprocessing plants, i.e. refineries, it is common to use a dewaxing reactor for the hydroprocessing of hydrocarbon feeds including renewable feeds such as vegetable oils into valuable hydrocarbon products in the transportation fuel range, such as diesel and jet fuel. The dewaxing sections comprising such reactors are normally more expensive in terms of capital and operating expenses (CAPEX and OPEX, respectively) than the section used for oligomerization of ethylene. It would therefore also be desirable to be able to provide a process and plant for producing a hydrocarbon stream which is capable of utilizing such already existing reactors (units).

### SUMMARY OF THE INVENTION

The present invention provides a process and plant to convert ethylene, optionally obtained from EtOH, for example from ethanol which is obtained by fermentation of biomass, i.e. from bio-ethanol, to SPK in high yield and with a low freezing point, while at the same time reducing the risk of fouling and thus plugging in the oligomerization of ethylene due to formation of wax and polymeric material.

Accordingly, in a first aspect, the invention provides, in a first general embodiment, a process for producing a hydrocarbon from ethylene according to the following steps:
a) converting an ethylene stream in an oligomerization reactor by contacting said ethylene stream with an oligomerization catalyst, said oligomerization catalyst being a homogeneous catalyst comprising:
a transition metal compound or complex comprising at least one of Ti, Zr, Cr, Ni, together with an alkyl aluminum compound dissolved in a hydrocarbon solvent or an etheral solvent; or
an alkyl aluminum compound dissolved in a hydrocarbon solvent, or an etheral solvent optionally as a co-catalyst;
for producing a first stream of linear alfa olefins (LAOs) in the C2-C20 range of which at least 80 wt % having a Schultz-Flory distribution with a chain propagation probability (alpha) of 0.35-0.60, or a Poisson distribution with a chain propagation probability (lambda) of 1.0-4.7;
b)
   b-1) separating said first stream of linear alfa olefins in the C2-C20 range into a second stream of C2-C6 olefins, and a third stream of C8+ olefins, such as C8-C20 olefins; and
   b-2) recycling at least a portion of the second stream of C2-C6 olefins from step b-1) to the oligomerization reactor.

The third stream, i.e. the LAO-stream of C8+ olefins, such as C8-C20 olefins, is suitably transported outside battery limits to a hydroprocessing plant, i.e. refinery, comprising a dewaxing section, and further processed therein.

In an embodiment, said hydrocarbon is synthetic paraffinic kerosene (SPK), and said process further comprises:
c) converting the third stream of C8+ olefins, such as C8-C20 olefins, step b-1) in the presence of hydrogen in a dewaxing reactor by contacting said third stream of C8+ olefins with a dewaxing catalyst, the dewaxing catalyst comprising: at least one transition metal selected from Ni, Pd, Pt or combinations thereof, and optionally another transition metal, and a zeolite or zeotype or a silica-alumina; and carrying out a dewaxing reaction comprising hydrogenation and isomerization of the olefins, for producing a fourth product stream of branched C8-C20 isoparaffins having a Branching Index (BI), defined as the average number of carbon atoms in the side chains divided by the average number of carbon atoms in the main chain, higher than 0.05;
d) separating the fourth product stream from step c) into a sixth stream of light end compounds, and a fifth product stream as synthetic paraffinic kerosene (SPK), said SPK having: i) a BI higher than 0.05, and ii) a freezing point below −40° C.

Suitably the freezing point is measured according to ASTM D2386.

As used herein, the term “first aspect” or “first aspect of the invention” refers to a process according to the invention. The term “second aspect” or “second aspect of the invention” refers to a plant (system) according to the invention. The term “another aspect of the invention” refers to a SPK product according to the invention. The term “yet another aspect of the invention” refers to a method of retrofitting an existing hydroprocessing plant for producing hydrocarbon products such as any of gasoline, jet fuel, diesel.

As used herein, the reference to specific compounds in a given stream means a stream rich in said compound or compounds. For instance, the term “ethylene stream” means a stream rich in ethylene. For instance, the term “first stream of linear alfa olefins in the C2-C20 range” means a stream rich in C2-C20 linear alfa olefins. For instance, the term “second stream of C4-C6 olefins” means a stream rich in C4-C6 olefins. The term “rich in a compound or compounds”, for instance a stream rich in C2-C6 olefins means more than 50 wt % of the stream, such as 60, 70, 80, 90 or 100 wt % of the stream.

As used herein, the terms “compound” and “component” may be used interchangeably.

As used herein, the term C8+ means hydrocarbons of up to 8 carbon atoms, optionally more than 8 carbon atoms; for instance C8-C20. For instance, C8+ olefins comprises C8-C20 olefins.

As used herein, the term “at least a portion” of a given stream means a portion of the stream or the entire stream.

As used herein, the term “synthetic paraffinic kerosene (SPK)”, means a mixture of alkanes, mostly iso-alkanes, in the C8-C18 range, suitably in accordance with ASTM D7566 Annex 5 where the ethylene is produced from renewable sources.

As used herein, and as already recited above, the term Branching Index (BI) is defined as the average number of carbon atoms in the side chains divided by the average number of carbon atoms in the main chain of hydrocarbons.

As used herein, the term “comprising” includes also “comprising only”, i.e. “consisting of”.

As used herein, the term “suitably” means “optionally” i.e. an optional embodiment.

As used herein, the use of the article “a” or “an” before an item or unit means “one or more”. For instance, the term “a hydrocarbon” means one or more hydrocarbons; the term “an oligomerization reactor” means one or more oligomerization reactors operating as the oligomerization reactor; such as one or more oligomerization reactors arranged in parallel.

As used herein, the term “unit” or “section” means a physically delimited space. A unit or section may comprise one or more units. For instance, a section may comprise one or more units. For instance, a unit such a fractionation unit may comprise one or more units, here one or more fractionation units. A reactor, such as dewaxing reactor, may also be regarded as a unit.

As used herein, a step may comprise one or more sub-steps. For instance, said step b) comprises step b-1) and b-2).

As used herein, the term “present invention” or “invention” may be used interchangeably with the term “present application” or “application”, respectively.

Other definitions are provided in connection with one or more embodiments.

The invention provides a process for converting ethylene, optionally obtained by dehydration of EtOH, to SPK in at least 75% overall yield and with a freezing point of −40° C. or below, or even −47° C. or below.

Accordingly, in an embodiment, the overall yield from ethylene in said ethylene stream of step a), to SPK in said seventh stream of step e), is at least 75% on a mole carbon basis, such as at least 80%, at least 85%, or at least 90%. The overall yield, herein also interchangeably referred to as “yield”, is defined as the ratio of the number of carbon atoms in the SPK with respect to the number of carbon atoms in the ethylene being fed to the first oligomerization reactor. The freezing point is of particular importance for jet fuel compositions since airplanes operate at high altitude and therefore at low temperature. The SPK thus obtained may be blended with hydrocarbons from other sources or used alone as jet fuel.

Existing processes for producing SPK from ethylene suffer from lower yields and/or higher freezing points. Furthermore, the invention enables reduced formation of wax and polymeric material in the oligomerization, due to the relatively low chain propagation probability, i.e. the chain propagation probability defined by: the “alpha-value”, or interchangeably “a-value” of the Schultz-Flory distribution of products being in the range of 0.35-0.60; or the “lambda-value”, or interchangeably, “A-value” of the Poisson distribution of products in the range of 1.0-4.7.

The invention provides a high yielding process with an SPK composition particularly rich in branched isomers, where the BI is above 0.05. Relevant isomers are for example the branched components 2-methyl-heptadecane (BI=0.059) and 2,3-dimethylhexadecane (BI=0.125). The high degree of branching and the general low symmetry of the isomers has the effect of decreasing the melting point (and thus, freezing point). Consider for example n-dodecane (BI=0), C₁₂H₂₆, which has a melting point of −10° C. The isomeric 2-methylundecane (BI=0.091) has a melting point of −47° C. while another isomer, 3-methylundecane (BI=0.091) has a melting point of 58° C.

The process layout allows to maximize yield of SPK in the fifth product stream by tuning the LAO distribution in the first stream from the oligomerization reactor.

The invention comprises the recycle of said second stream of C2-C6 olefins to the oligomerization reactor (step b-2)), thereby enabling to build up longer LAOs. There are other associated benefits thereto: this solves the problem of the LAOs being too light for SPK, thus for use as jet fuel, and increases the overall SPK yield; there is a higher degree of branching upon recycling, which is normally unwanted, yet as a feedstock for SPK it is desirable; in case a low boiling hydrocarbon solvent is used in the oligomerization reactor, such as benzene, this can also be recycled back without the need to separate it from, e.g. the C8 LAO. Optionally, a portion of the second stream of C2-C6 olefins is used for making aromatics.

In an embodiment, as also recited farther below, the hydrocarbon solvent is benzene. This is highly advantageous, as benzene can then be recycled together with the second stream of C2-C6 olefins.

It would be understood, that in step a), the term “for producing a first stream of linear alfa olefins (LAOs) in the C2-C20 range of which at least 80 wt % having a Schultz-Flory distribution with a chain propagation probability (alpha) of 0.35-0.60, or a Poisson distribution with a chain propagation probability (lambda) of 1.0-4.7, means that 80 wt % or more, such as 90 wt % or 100 wt % of the C2-C20 LAOs have said distribution. As is well-known in the art, the chain propagation probability (alpha) in a Schultz-Flory distribution of products stems from the equation ωₐ(k)=a² k(1−a)^{k-1}, which is the probability weight function of the weight fraction of k-length chains. Kappa “k” is the number of monomers in the chain, here denoted as the alfa-olefin carbon number, and alfa “a” which has a value between 0 and 1, measures said chain propagation probability. The lower the alpha value, the more the shorter chains are favored. See for instance appended FIG. 2.

As is well-known in the art, the chain propagation probability (lambda) in a Poison distribution of products stems from the equation f(x)=(e^{-λ}λ^{x})/x!, where x is the number of occurrences and lambda “λ” is the expected rate of occurrences, here denoted as the alfa-olefin carbon number. The lower the lambda value, the more the shorter chains are favored. For the purposes of the present application, lambda measures the chain propagation probability. The molar fraction of the products is suitably described by the Poisson distribution and then converted to the corresponding weight fraction. See for instance appended FIG. 3.

In an embodiment, in b-1) said separating step is by fractionation in a fractionation unit, for instance distillation such as flash distillation in a flash distillation column, or in a distillation column.

In an embodiment, in step a) the chain propagation probability is 0.40-0.54, such as 0.45 for a Schulz-Flory distribution; or 2.5-4.0 such as 3.1 for a Poisson distribution.

By the invention, ethylene is oligomerized to produce a distribution of linear alpha olefins (LAOs) in the range C2-C20 (C2=-C20=), for instance comprising less than 5 wt %, such as 1 wt % C20+, or such as 0.5 wt % C20+. This distribution can be a Schulz-Flory distribution with an alfa-value in the range 0.35-0.60, such as 0.40-0.54, preferably 0.45 (see FIG. 2) which gives an approximate composition of the obtained first product stream (first stream of LAOs) of 30 wt % unconverted C2=, 45% C4=-C6=, 24% C8=-C18= and 0.5% C20+=. The distribution of LAOs could also be a Poisson distribution with lambda in range 1.0-4.7, such as 2.5-4.0, preferably 3.1 (see FIG. 3), which gives an approximate composition of the obtained first product stream of 3 wt % unconverted C2=, 29 wt % C4=-C6=, 67 wt % C8=-C18= and 1 wt % C20+=. Accordingly, the operation with the particular values of alfa (0.45) or lambda (3.1) enables minimizing the production of C2= and C20+=.

In an embodiment, said SPK, i.e. the fifth product stream, has a freezing point of −47° C. or below and/or a BI of up to 0.150.

The SPK comprises for instance a combination of any of the branched components: 2-methyl-heptadecane (BI=0.059), 2,3-dimethylhexadecane (BI=0.125) and 2-methylundecane (BI=0.091) and 3-methylundecane (BI=0.091). Hence, there is a high degree of branching combined with low symmetry in the distribution of isomers. The high degree of branching and the general low symmetry of the isomers has the effect of decreasing the melting point and thereby the freezing point. Consider again for example n-dodecane (BI=0), C₁₂H₂₆, which has a melting point of −10° C. The isomeric 2-methylundecane (BI=0.091) has a melting point of −47° C., while the isomer 3-methylundecane (BI=0.091) has a melting point of −58° C.

In an embodiment, the process further comprises, prior to said step a), a step of catalytic dehydration of ethanol for producing said ethylene stream; optionally the process further comprising fermentation of biomass for producing at least a portion of said ethanol.

A sustainable approach for producing SPK from a renewable source, biomass, is thereby achieved.

The ethanol may be provided by different sources, for instance from the fermentation of biomass, in which carbohydrates are converted to ethanol and carbon dioxide by microorganisms. The biomass is for instance a lignocellulosic biomass or non-lignocellulosic biomass. The term “lignocellulosic biomass” means a biomass containing, cellulose, hemicellulose and optionally also lignin. The lignin or a significant portion thereof may have been removed, for instance by a prior bleaching step. The biomass can be forestry waste and/or agricultural residue and comprises biomass originating from plants including grass such as nature grass (grass originating from natural landscape), wheat e.g. wheat straw, oats, rye, reed grass, bamboo, sugar cane or sugar cane derivatives such as bagasse, maize and other cereals. The biomass may thus include starch containing materials such as wheat and corn. The biomass may also include sucrose containing materials such as sugar beet and sugar cane.

In an embodiment, the process further comprises: supplying at least a portion of said sixth stream of light end compounds to a hydrogen producing unit (HPU) or a gasification unit (GU) for providing a make-up hydrogen stream or a synthesis gas stream; and providing at least a portion of said make-up hydrogen gas as said hydrogen in said step c) i.e. dewaxing.

The sixth stream of light end compounds, herein also referred to as light end stream, comprises for instance C3-C4 compounds such as LPG. The light end stream may also comprise naphtha compounds, such as C5-C9 hydrocarbons. The light end stream may comprise one or more side streams generated in the process or plant such as off-gases containing some C1-C2 hydrocarbons. While some of the light end stream(s) may then be withdrawn as an off-gas and used as fuel in the process or plant, for instance in burners of fired heaters or auxiliary boilers for producing steam, at least a portion, suitably a major portion, or the entire portion, is used to generate make-up hydrogen used in the process or plant, thereby enabling high integration in the process as the required hydrogen is internally sourced instead of being externally sourced.

In another embodiment, a portion of the light end stream is withdrawn as a separate naphtha stream. This can then be sold as a separate product.

The HPU comprises a steam reforming unit. The steam reforming unit is for instance an electrically heated steam methane reformer (e-SMR), optionally with a pre-reformer arranged upstream. The steam reforming unit is for instance an autothermal reformer (ATR), optionally with a pre-reformer arranged upstream. The steam reforming unit is for instance a convection reformer, optionally with a pre-reformer arranged upstream. Combinations of the above are also envisaged. The HPU may further comprise a water gas shift (WGS) section as well as a hydrogen purification unit such as a pressure swing adsorption (PSA) unit, for producing said make-up hydrogen gas. The HPU may further comprise a CO₂-removal unit, for instance in between the WGS section and the hydrogen purification unit. The removed CO₂ rich gas may then be subjected to carbon capture, utilization and storage (CCUS) according to known techniques.

Now more specifically, in an embodiment, the steam reforming unit is: a convection reformer, preferably comprising one or more bayonet reforming tubes such as an HTCR reformer i.e. Topsøe bayonet reformer, where the heat for reforming is transferred by convection along with radiation; a tubular reformer i.e. conventional steam methane reformer (SMR), where the heat for reforming is transferred chiefly by radiation in a radiant furnace; autothermal reformer (ATR), where partial oxidation of the hydrocarbon feed with oxygen and steam followed by catalytic reforming; electrically heated steam methane reformer (e-SMR), where electrical resistance is used for generating the heat for catalytic reforming; or combinations thereof. In particular, when using e-SMR, electricity from green resources may be utilized, such as from electricity produced by wind power, hydropower, and solar sources, optionally from thermonuclear power, thereby further minimizing the carbon footprint. Furthermore, while a convection reformer, a SMR, or an ATR will require the burning of a great portion of e.g. LPG for producing heat for reforming, an e-SMR enables full utilization of the LPG or any other off-gas for the reforming and then production of the make-up hydrogen gas. Hence, there is a better utilization and conversion of light end compounds to the desired make-up hydrogen gas.

For more information on these reformers, details are herein provided by direct reference to Applicant's patents and/or literature. For instance, for tubular and autothermal reforming an overview is presented in “Tubular reforming and autothermal reforming of natural gas—an overview of available processes”, Ib Dybkjær, Fuel Processing Technology 42 (1995) 85-107; and EP 0535505 for a description of HTCR. For a description of e-SMR, reference is given to in particular WO 2019/228797 A1.

In an embodiment, the catalyst in the steam reforming unit is a reforming catalyst, e.g. a nickel based catalyst. In an embodiment, the catalyst in the water gas shift reaction is any catalyst active for water gas shift reactions. The said two catalysts can be identical or different. Examples of reforming catalysts are Ni/MgAl₂O₄, Ni/Al₂O₃, Ni/CaAl₂O₄, Ru/MgAl₂O₄, Rh/MgAl₂O₄, Ir/MgAl₂O₄, MO₂C, WO₂C, CeO₂, Ni/ZrO₂, Ni/MgO—Al₂O₃, Ni/CaAl₂O₄, Ru/MgO—Al₂O₃, or Rh/MgO—Al₂O₃, a noble metal on an Al₂O₃ carrier, but other catalysts suitable for reforming are also conceivable. The catalytically active material may be Ni, Ru, Rh, Ir, or a combination thereof, while the ceramic coating may be Al₂O₃, ZrO₂, MgAl₂O₄, CaAl₂O₄, or a combination therefore and potentially mixed with oxides of Y, Ti, La, or Ce. The maximum temperature of the reactor may be between 850-1300° C. The pressure of the feed gas may be 15-180 bar, preferably about 25 bar. Steam reforming catalyst is also denoted steam methane reforming catalyst or methane reforming catalyst.

The HPU may also produce a synthesis gas (syngas) stream, for instance by withdrawing a stream downstream the steam reforming unit, for instance from between the steam reforming unit and the WGS section, or from the WGS section, or from between the WGS section and the hydrogen purification unit, e.g. PSA unit.

The at least a portion of said sixth stream of light end compounds may also be supplied to a gasification unit (GU) for providing the syngas.

The GU as is well-known in the art syngas production, is a unit that converts a carbonaceous material at temperatures above 700° C. without combustion with a controlled amount of oxygen and/or steam.. It is to be understood, that the GU may also include a water gas shift (WGS) reactor downstream the gasifier so as to convert CO with water to H₂ and CO₂, thus increasing the yield of the make-up hydrogen stream.

WGS, PSA and GU are well-known technologies in the art of syngas and hydrogen production.

As is also well-known in the art, the syngas is a mixture of CO and H₂, often in combination with CO₂ and/or H₂O. As used herein, the term syngas is to be understood in its broadest sense as any mixture of CO, CO₂, H₂ and H₂O, possibly also containing gases which are inert to the reaction conditions such as Ar, N₂ and CH₄.

In an embodiment, said step c) is carried out by sequential hydrogenation and dewaxing, by said third stream of C8+ olefins, such as C8-C20 olefins, being first hydrogenated in a first hydrogenation zone and subsequently isomerized in a dewaxing zone.

This enables better control of the dewaxing (hydroisomerization) and thus of the resulting BI of fifth product stream of branched C8-C20 isoparaffins, which after the subsequent separation, e.g. fractionation, of step d) produces the desired SPK.

As used herein, the term dewaxing and hydroisomerization are used interchangeably.

The dewaxing zone is arranged downstream the hydrogenation zone. The first hydrogenation zone is for instance a hydrogenation reactor. The dewaxing zone is for instance a dewaxing reactor arranged downstream the hydrogenation reactor. The first hydrogenation zone and the dewaxing zone are for instance provided as different catalytic fixed beds in the same reactor.

In an embodiment, the dewaxing catalyst comprises at least one transition metal selected from Ni, Pd, Pt and optionally another transition metal, and a zeolite or zeotype or a silica-alumina, to carry out a dewaxing reaction comprising hydrogenation and isomerization of the olefins to yield the fourth product stream of mainly branched C8-C20 isoparaffins.

The material catalytically active in dewaxing typically comprises an active metal (either elemental noble metals such as platinum and/or palladium or sulfided base metals such as nickel, cobalt, tungsten and/or molybdenum), an acidic support (typically a molecular sieve showing high shape selectivity, and having a topology such as MOR, FER, MRE, MWW, AEL, TON and MTT) and a refractory support (such as alumina, silica or titania, or combinations thereof).

The dewaxing conditions involve typically a temperature in the interval 250-400° C., a pressure in the interval 20-100 bar, and a liquid hourly space velocity (LHSV) in the interval 0.5-8.

Suitably, excess hydrogen from dewaxing reactor is produced, and the process further comprises supplying at least a portion of said excess hydrogen to a hydrogen purification unit, such as a PSA unit, suitably to the PSA unit of said hydrogen producing unit (HPU).

Thereby, further integration is provided as make-up hydrogen gas from the HPU is not only produced from the light end stream, but the HPU particularly the hydrogen purification unit therein e.g. PSA unit, serves to purify any excess hydrogen streams produced in the process. The HPU can be made smaller, thus requiring i.a. smaller plot size. The make-up hydrogen gas is recycled to the dewaxing reactor, thus further enabling the process being self-sufficient with hydrogen. Where an e-SMR is provided in the HPU, a significantly low carbon footprint is also possible, as the e-SMR is powered by electricity produced from renewable sources such as wind and solar, optionally from thermonuclear power.

In an embodiment, in any of step a) or c-1):
the oligomerization catalyst is a dissolved metal catalyst, in which the metal is said transition metal, and the transition metal is present in the concentration range 0.01-1.0 wt %; and/or
said alkyl aluminum catalyst is an alkyl aluminum compound such as triethyl aluminum or methyl aluminumoxide (MAO).

In an embodiment, the oligomerization catalyst is a combination of titanium or zirconium alkoxides or carboxylates with alkyl aluminum compounds such as MAO; or complexes of Cr or Ni in combination with alkyl aluminum compounds such as MAO. The alkyl aluminum catalyst acts suitably as a co-catalyst.

Hence, the oligomerization catalyst is for instance an alkyl aluminum compound such as triethyl aluminum, or a combination of titanium or zirconium alkoxides or carboxylates with alkyl aluminum compounds such as MAO or complexes of Cr or Ni in combination with MAO or other alkyl aluminum compounds. In general, it has been found that the oligomerization is better catalyzed by a transition metal compound in combination with an alkyl aluminum compound. The chain propagation probability in the first oligomerization reaction is suitably controlled by the ratio between these two catalyst components and/or by the process conditions in terms of temperature and pressure, as for instance described in S. A. Svejda and M. Brookhart, Organometallics 18 (1999), 65-74, for a Schultz-Flory distribution; or as for instance described in WO 2007090412 for a Poisson distribution.

In an embodiment, the oligomerization in any of steps a) or c-1) is carried out at a temperature in the range 0-250° C., such as 20-200° C. and at a pressure in the range 0.5-30 MPa, such as 1-15 MPa. For instance, the temperature is 40, 60, 80, 100, 120, 140, 160, 180° C. For instance, the pressure is 5, 10, 15, 20, 25 MPa. The pressure values are absolute pressure.

In an embodiment, the hydrocarbon solvent of the oligomerization catalyst is any of: benzene, toluene, a portion of fifth product stream i.a. a portion of the SPK, a portion of the fourth product stream i.e. isoparaffins in the C8-C20 range, a combination of the fifth and fourth product streams. Combinations of any of these streams is also envisaged.

The oligomerization is thus carried out in appropriate solvents which can be for example benzene, toluene, an ethereal solvent, or product streams produced in the process. Further integration of process streams is thereby provided.

Hence, for instance, in an embodiment, the hydrocarbon solvent is benzene. Benzene boils at 80° C., just in between the LAO's C6=(63° C.) and C8= (121° C.). This is highly advantageous, as benzene can then be recycled together with the second stream of C2-C6 olefins; for instance, by admixing a benzene stream with the second stream of C2-C6 olefins.

In an embodiment, the etheral solvent of the oligomerization catalyst is an ether, such as any of: diethyl ether, dipropyl ether, tetrahydrofuran, 1,4-dioxane or tetrahydropyran, or combinations thereof.

In an embodiment, prior to the separation step b-1):
the first stream of LAOs is quenched by admixture with a solvent capable of neutralizing the dissolved catalyst species and removing them from the hydrocarbon phase of said first stream of LAOs, in which the solvent is a polar solvent comprising water, an alcohol, or a combination thereof;
optionally the dissolved catalyst or catalysts are separated from the first stream of LAOs and recycled to the oligomerization reactor.

In an embodiment, said SPK, i.e. the fifth product stream, comprises less than 1 pppm wt of any of Al, Ni, Cr, and combinations thereof. Suitably also, the first product stream of LAOs comprises more Al and/or Cr than said SPK.

In an embodiment, said SKP, i.e. the fifth product stream, further comprises >65 wt % paraffins, such as >95 wt %, (with said BI>0.05), a specific gravity in the range 0.73-0.77 g/cm³ as measured by ASTM D287. Hence, apart from the already recited BI>0.05 and freezing point below −40° C., the SPK has the required density and is highly paraffinic.

In another embodiment, the LAOs in the first stream are not necessarily limited to any Schultz-Flory or Poisson distribution. Accordingly, in a second general embodiment of the first aspect of the invention, there is also provided a process for producing a hydrocarbon from ethylene according to the following steps:
a) converting an ethylene stream in an oligomerization reactor by contacting said ethylene stream with an oligomerization catalyst having a selectivity for trimerization and tetramerization of LAO's of at least 70%, said oligomerization catalyst being a homogeneous catalyst comprising:
a transition metal compound or complex comprising at least one of Ti, Zr, Cr, Ni, together with an alkyl aluminum compound dissolved in a hydrocarbon solvent or an etheral solvent; or
an alkyl aluminum compound dissolved in a hydrocarbon solvent or an etheral solvent, optionally as a co-catalyst;
for producing a first stream of linear alfa olefins (LAOs) in the C2-C20 range; optionally, wherein at least 80 wt% having a Schultz-Flory distribution with a chain propagation probability (alpha) of 0.35-0.60, or a Poisson distribution with a chain propagation probability (lambda) of 1.0-4.7;
b)
   b-1) separating said first stream of linear alfa olefins in the C2-C20 range into a second stream of C2-C6 olefins, and a third stream of C8+ olefins, such as C8-C20 olefins; and
   b-2) recycling at least a portion of the second stream of C2-C6 olefins from step b-1) to the oligomerization reactor.

A narrow distribution of LAOs in the first stream is thereby obtained, such as a high proportion of C8-C18 olefins, thus already within the desired range of SPK. There is also high yield of such C8-C18 olefins following the separation in step b), i.e. in said third stream. Thereby also, there is need for less recycling of the second stream of C2-C6 olefins, as said high proportion of C8-C18 olefins, already after the oligomerization and separation, provides a high weight ratio of the third stream with respect to the second stream.

Any of the above process embodiments may be used in connection with this second general embodiment.

More generally, in another aspect of the invention, there is also provided a SPK product obtainable by the process as defined in any of claims 2-16, comprising: >65 wt % paraffins, such as >95 wt %, a BI>0.05, e.g. BI>0.05 and up to 0.150, a specific gravity in the range of 0.73-0.77 g/cm³ as measured by ASTM D287, and a freezing point below −40° C., e.g. −47° C., as measured by ASTM D2386; optionally wherein the SPK product is in compliance with ASTM D7566-Annex 5.

As recited farther above, the term “synthetic paraffinic kerosene (SPK)”, means a mixture of alkanes, mostly iso-alkanes (iso-paraffins), in the C8-C18 range. The high content of paraffins together with BI>0.05, particularly in said range up to 0.150, along with the specific gravity (density) enables the provision of a superior SPK product. The SPK product is suitably in accordance with ASTM D7566-Annex 5 where the ethylene is produced from renewable sources e.g. biomass. The SPK may thus be a sustainable aviation fuel (SAF) in accordance with ASTM D7566-Annex 5 and it may then be blended with conventional jet fuel to a maximum limit of 50 vol %, thereby in compliance with ASTM D1655 corresponding to conventional Jet A or Jet A1 kerosene.

In a second aspect of the invention, there is also envisaged a plant (system) for carrying out the process of the invention.

Accordingly, there is also provided a plant for carrying out the process according to any of the above process embodiments, comprising:
an oligomerization reactor arranged to receive: an ethylene stream and at least a portion of a second stream of C2-C6 olefins from a downstream first separation section; the oligomerization rector comprising an oligomerization catalyst, said oligomerization catalyst being a homogeneous catalyst comprising: a transition metal compound or complex comprising at least one of Ti, Zr, Cr, Ni, together with an alkyl aluminum compound dissolved in a hydrocarbon solvent or an etheral solvent; or an alkyl aluminum compound dissolved in a hydrocarbon solvent or an etheral solvent, optionally as a co-catalyst; the oligomerization reactor further being arranged to provide a first stream of linear alfa olefins (LAOs) in the C2-C20 range of which at least 80 wt % having a Schultz-Flory distribution with a chain propagation probability (alpha) of 0.35-0.60, or a Poisson distribution with a chain propagation probability (lambda) of 1.0-4.7;
said first separation section arranged to receive said first stream of LAOs and to provide: said second stream of C2-C6 olefins, and a third stream of C8+ olefins, said first separation section further comprising a conduit arranged for recycling said at least a portion of second stream of C2-C6 olefins to the oligomerization reactor;
optionally, a dewaxing reactor arranged to receive the third stream of C8+ olefins as well as hydrogen, the dewaxing reactor comprising a dewaxing catalyst, the dewaxing catalyst comprising: at least one transition metal selected from Ni, Pd, Pt or combinations thereof, and optionally another transition metal, and a zeolite or zeotype or a silica-alumina; the dewaxing reactor further being arranged to provide a fourth product stream of branched C8-C20 isoparaffins;
optionally, a second separation section arranged to receive said fourth product stream of branched C8-C20 isoparaffins; and to provide: a sixth stream of light end compounds, and a fifth product stream as synthetic paraffinic kerosene (SPK), and suitably wherein said SPK has: i) a Branching Index, BI, defined as the average number of carbon atoms in the side chains divided by the average number of carbon atoms in the main chain, higher than 0.05, and ii) a freezing point below −40° C.

In yet another aspect of the invention, there is also provided a method of retrofitting an existing hydroprocessing plant, said hydroprocessing plant comprising: a dewaxing reactor, i.e. an existing dewaxing reactor and a separation section arranged downstream said dewaxing reactor, i.e. an existing separation section; the method of retrofitting comprising:
installing an oligomerization reactor upstream said dewaxing reactor, in which the oligomerization reactor is arranged to receive: an ethylene stream and at least a portion of a second stream of C2-C6 olefins from a downstream first separation section; the oligomerization rector comprising an oligomerization catalyst, said oligomerization catalyst being a homogeneous catalyst comprising: a transition metal compound or complex comprising at least one of Ti, Zr, Cr, Ni, together with an alkyl aluminum compound dissolved in a hydrocarbon solvent or an etheral solvent; or an alkyl aluminum compound dissolved in a hydrocarbon solvent or an etheral solvent, optionally as a co-catalyst; the oligomerization reactor further being arranged to provide a first stream of linear alfa olefins (LAOs) in the C2-C20 range of which at least 80 wt % having a Schultz-Flory distribution with a chain propagation probability (alpha) of 0.35-0.60, or a Poisson distribution with a chain propagation probability (lambda) of 1.0-4.7;
installing said first separation section downstream and in direct fluid communication with said oligomerization reactor, in which the first separation section is arranged to receive said first stream of LAOs and to provide: said second stream of C2-C6 olefins, and a third stream of C8+ olefins;
installing a conduit in direct fluid communication between the first separation section and the oligomerization reactor, for recycling said at least a portion of second stream of C2-C6 olefins from the first separation section to the oligomerization reactor;
installing a conduit in direct fluid communication between the first separation section and said dewaxing reactor, which is arranged downstream, for supplying said third stream of C8+ olefins to the dewaxing reactor, and in which the dewaxing reactor is further arranged to provide a fourth product stream of branched C8-C20 isoparaffins;
installing a conduit in direct fluid communication between the dewaxing reactor and said separation section arranged downstream said dewaxing reactor for supplying said fourth product stream of branched C8-C20 isoparaffins to said separation section; said separation section is further arranged to provide: a sixth stream of light end compounds; and a fifth product stream as synthetic paraffinic kerosene (SPK), said SPK suitably having: i) a Branching Index, BI, defined as the average number of carbon atoms in the side chains divided by the average number of carbon atoms in the main chain, higher than 0.05, and ii) a freezing point below −40° C.;
optionally, installing a hydrogen producing unit (HPU) or a gasification unit (GU) in direct or indirect fluid communication with said separation section arranged downstream said dewaxing reactor; said HPU being arranged to receive at least a portion of the sixth stream of light end compounds and provide a make-up hydrogen stream;
the method further comprising installing a conduit for providing at least a portion of said make-up hydrogen gas to said dewaxing reactor.

In a particular embodiment of the yet another aspect of the invention, there is provided a method of retrofitting an existing hydroprocessing plant, said hydroprocessing plant comprising: a dewaxing reactor and a separation section arranged downstream said dewaxing reactor; the method of retrofitting comprising the provision of a plant as defined in any of the above plant embodiments, by:
installing an oligomerization reactor upstream said dewaxing reactor, in which the oligomerization reactor is arranged to receive: an ethylene stream and at least a portion of a second stream of C2-C6 olefins from a downstream first separation section; the oligomerization rector comprising an oligomerization catalyst, said oligomerization catalyst being a homogeneous catalyst comprising: a transition metal compound or complex comprising at least one of Ti, Zr, Cr, Ni, together with an alkyl aluminum compound dissolved in a hydrocarbon solvent or an etheral solvent; or an alkyl aluminum compound dissolved in a hydrocarbon solvent or an etheral solvent, optionally as a co-catalyst; the oligomerization reactor further being arranged to provide a first stream of linear alfa olefins (LAOs) in the C2-C20 range of which at least 80 wt % having a Schultz-Flory distribution with a chain propagation probability (alpha) of 0.35-0.60, or a Poisson distribution with a chain propagation probability (lambda) of 1.0-4.7;
installing said first separation section downstream and in direct fluid communication with said oligomerization reactor, in which the first separation section is arranged to receive said first stream of LAOs and to provide: said second stream of C2-C6 olefins, and a third stream of C8+ olefins;
installing a conduit in direct fluid communication between the first separation section and the oligomerization reactor, for recycling said at least a portion of second stream of C2-C6 olefins from the first separation section to the oligomerization reactor;
installing a conduit in direct fluid communication between the first separation section and said dewaxing reactor, which is arranged downstream, for supplying said third stream of C8+ olefins to the dewaxing reactor, and in which the dewaxing reactor is further arranged to provide a fourth product stream of branched C8-C20 isoparaffins;
installing a conduit in direct fluid communication between the dewaxing reactor and said separation section arranged downstream said dewaxing reactor for supplying said fourth product stream of branched C8-C20 isoparaffins to said separation section; said separation section is further arranged to provide: a sixth stream of light end compounds; and a fifth product stream as synthetic paraffinic kerosene (SPK), said SPK suitably having: i) a Branching Index, BI, defined as the average number of carbon atoms in the side chains divided by the average number of carbon atoms in the main chain, higher than 0.05, and ii) a freezing point below −40° C.;
optionally, installing a hydrogen producing unit (HPU) or a gasification unit (GU) in direct or indirect fluid communication with said separation section arranged downstream said dewaxing reactor; said HPU being arranged to receive at least a portion of the sixth stream of light end compounds and provide a make-up hydrogen stream;
the method further comprising installing a conduit for providing at least a portion of said make-up hydrogen gas to said dewaxing reactor.

It would be understood that in this yet another aspect of the inventio, the dewaxing reactor is the existing dewaxing reactor, i.e. the dewaxing reactor of the existing hydrocarbon plant. It would be understood that the separation section arranged downstream said dewaxing reactor, i.e. the separation section of the existing hydrocarbon plant. This separation section corresponds to the second separation section of the process and plant according to the invention.

As used herein, the term “in direct fluid communication” means that there are no intermediate units or process steps significantly changing the composition of the stream.

As used herein, the term “in indirect fluid communication” means that there are intermediate units or process steps significantly changing the composition of the stream.

As used herein, the term “hydroprocessing plant” may be used interchangeably with the term “refinery”.

By this method, it is possible to benefit from existing infrastructure of a hydroprocessing plant. The dewaxing reactor as well as the downstream separation section are common in hydroprocessing plants, yet these, in particular the dewaxing reactor are the more costly parts of the units and associated equipment required for the producing of SPK from ethylene. The LAO front-end, hence the oligomerization reactor and first separation section are for instance installed at or nearby such hydroprocessing plants; or alternatively, a LAO stream (C8-C20 olefins) corresponding to said third stream of C8+ olefins, such as the C8-C20 olefins, is transported to the hydroprocessing plant(s).

Any of the embodiments and associated benefits of the first aspect of the invention may be used in the second aspect of the invention or the another aspect of the invention, or the yet another aspect of the invention, or vice versa.

### BRIEF DESCRIPTION OF DRAWINGS

FIG. 1 is a schematic layout of the process and plant according to an embodiment of the invention.

FIG. 2 is a graph showing the product distribution obtained in the first oligomerization step, i.e. the first stream of LAOs, for three different values of alfa, given a Schulz-Flory product distribution.

FIG. 3 is a graph showing the product distribution obtained in the first oligomerization step, i.e. the first stream of LAOs, for three different values of lambda, given a Poisson product distribution.

FIG. 4 is an example of product distribution from the conversion of n-C18 in a Dewaxing reactor, i.e. the seventh product stream.

### DETAILED DESCRIPTION

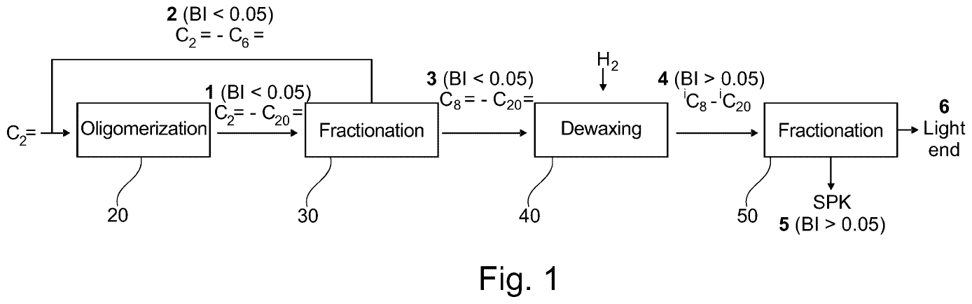

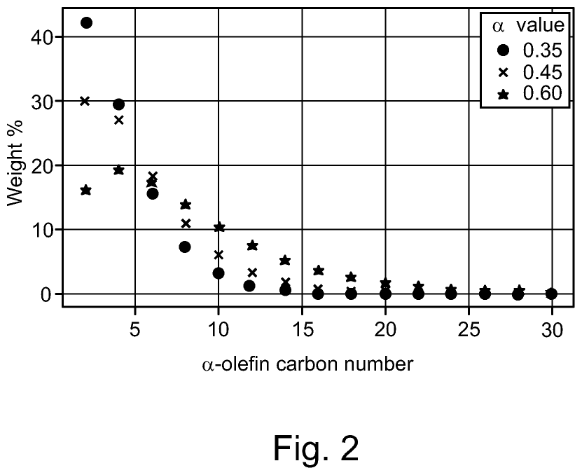

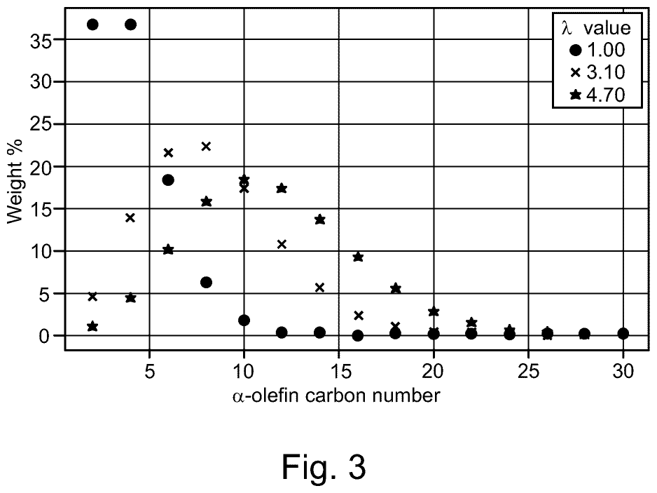

With reference to FIGS. 1, 2 and 3. In FIG. 1, ethylene (C2=) is oligomerized in oligomerization reactor 20 to produce a first stream (first product stream) 1 having a distribution of linear alpha olefins (LAOs) in the range C2=-C20=. For instance, the LAOs have less than 5 wt %, such as 1 wt % C20+ or such as 0.5 wt % C20+. This distribution is a Schulz-Flory distribution with an alfa-value in the range 0.35-0.60, such as 0.40-0.54, preferably 0.45 (see FIG. 2) which gives an approximate composition of the obtained first product stream 1 of 30 wt % unconverted C2=, 45% C4=-C6 =, 24% C8=-C18= and 0.5% C20+=. The distribution may also be a Poisson distribution with lambda parameter in the range 1.0-4.7, such as 2.5-4.0, such as 3.1 (see FIG. 3), which gives an approximate composition of the obtained first product stream of 3 wt % unconverted C2=, 29 wt % C4=-C6 =, 67 wt % C8=-C18= and 1 wt % C20+=. This first product stream 1 is separated, e.g. fractionated, in a first separation section 30 into: second stream 2 of C2-C6 olefins with BI>0.05 which is recycled to the oligomerization (oligomerization reactor), and a third stream 3 of C8+ olefins, such as C8-C20 olefins. This third stream 3 is converted in a dewaxing reactor 40 together with hydrogen to a fourth stream 4 of branched C8-C20 isoparaffins, thus mainly isoparaffinic C8-C18, having a BI>0.05. The fourth stream 4 comprises for instance at least 80% by weight of branched (BI >0.05) isoparaffins in the C8-C20 range. This fourth stream 4 is then fractionated in second separation section 50 into: a fifth product stream 57 as SPK product, having a BI>0.05 and up to 0.150, a specific gravity in the range 0.73-0.77 g/cm³; said sixth stream of light end compounds 6, i.e. light end stream, which comprises low-boiling compounds such as LPG and/or naphtha. This light end stream 6 may also comprise other off-gases and may be used as fuel gas in the process. The light end stream 6 is suitably utilized to generate make-up hydrogen gas in a hydrogen producing unit (not shown) used in the process, such as the hydrogen for dewaxing. Part of light end stream 6 may also be sold as a separate product such as naphtha. Any excess hydrogen from the dewaxing reactor 40 is optionally purified by for example a PSA unit in the HPU (not shown) and recycled back to the process.

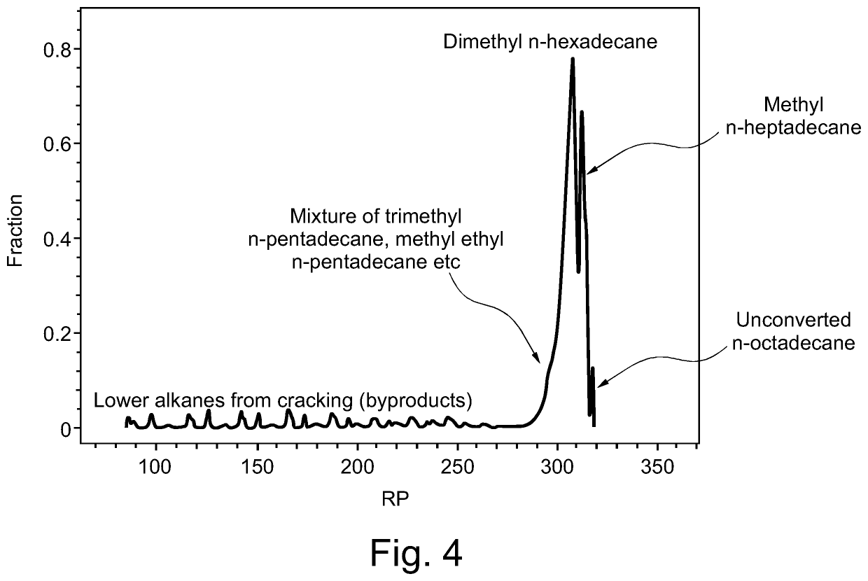

Now turning to FIG. 4, it is shown a gas chromatogram showing the effluent stream from conversion of n-octadecane with H₂ at 3.5 MPa at 340° C. over a commercially available noble metal loaded ZSM-48 as dewaxing catalyst. The graph shows the peaks corresponding to the formation of dimethyl n-hexadecane and methyl n-heptadecane.

1. A process for producing a hydrocarbon from ethylene, the process comprising:
a) converting an ethylene stream in an oligomerization reactor by contacting the ethylene stream with an oligomerization catalyst, the oligomerization catalyst being a homogeneous catalyst comprising:
a transition metal compound or complex comprising at least one of Ti, Zr, Cr, Ni, together with an alkyl aluminum compound dissolved in a hydrocarbon solvent or an etheral solvent; or
an alkyl aluminum compound dissolved in a hydrocarbon solvent or an etheral solvent, optionally as a co-catalyst;
for producing a first stream of linear alfa olefins (LAOs) in the C2-C20 range, of which at least 80 wt % have a Schultz-Flory distribution with a chain propagation probability (alpha) of from 0.35-0.60, or a Poisson distribution with a chain propagation probability (lambda) of from 1.0-4.7; and
b)
b-1) separating the first stream of linear alfa olefins in the C2-C20 range into a second stream of C2-C6 olefins, and a third stream of C8+olefins, and
b-2) recycling at least a portion of the second stream of C2-C6 olefins from step b-1) to the oligomerization reactor.

2. The process of claim 1, wherein the hydrocarbon is synthetic paraffinic kerosene (SPK), and the process further comprises:
c) converting the third stream of C8+ olefins, in the presence of hydrogen in a dewaxing reactor by contacting the third stream of C8+ olefins with a dewaxing catalyst, the dewaxing catalyst comprising at least one transition metal selected from Ni, Pd, Pt or combinations thereof, and optionally another transition metal, and a zeolite or zeotype or a silica-alumina; and carrying out a dewaxing reaction comprising hydrogenation and isomerization of the olefins, for producing a fourth product stream of branched C8-C20 isoparaffins having a Branching Index (BI), defined as the average number of carbon atoms in the side chains divided by the average number of carbon atoms in the main chain, higher than 0.05; and
d) separating the fourth product stream from step c) into a sixth stream of light end compounds, and a fifth product stream as synthetic paraffinic kerosene (SPK), the SPK having: i) a BI higher than 0.05, and ii) a freezing point below −40° C.

3. The process of claim 1, wherein the overall yield from ethylene in the ethylene stream of step a) to SPK in the fifth stream of step d) is at least 75% on a mole carbon basis.

4. The process of claim 1, wherein in step a) the chain propagation probability is from 0.40-0.54, for a Schulz-Flory distribution; or from 2.5-4.0 for a Poisson distribution.

5. The process of claim 2, wherein the SPK has a freezing point of −47° C. or below, and/or a BI of up to 0.150.

6. The process of claim 1, wherein the process further comprises, prior to the step a), a step of catalytic dehydration of ethanol for producing the ethylene stream; and optionally the process further comprising fermentation of biomass for producing at least a portion of the ethanol.

7. The process of claim 2, wherein the process further comprises: supplying at least a portion of the sixth stream of light end compounds to a hydrogen producing unit (HPU) or a gasification unit (GU) for providing a make-up hydrogen stream or a synthesis gas stream; and providing at least a portion of the make-up hydrogen gas as the hydrogen in the step c).

8. The process of claim 2, wherein step c) is carried out by sequential hydrogenation and dewaxing, by the third stream of C8+ olefins, being first hydrogenated in a first hydrogenation zone and subsequently isomerized in a dewaxing zone.

9. The process of claim 1, wherein in step a):
the oligomerization catalyst is a dissolved metal catalyst, in which the metal is the transition metal and the transition metal is present in a concentration range of 0.01-1.0 wt %; and/or
the alkyl aluminum catalyst is an alkyl aluminum.

10. The process of claim 1, wherein the oligomerization catalyst is a combination of titanium or zirconium alkoxides or carboxylates with alkyl aluminum compounds; or complexes of Cr or Ni in combination with alkyl aluminum compounds.

11. The process of claim 1, wherein the oligomerization in step a) is carried out at a temperature in the range of 0-250° C., and at a pressure in the range of 0.5-30 MPa.

12. The process of claim 2, wherein:
the hydrocarbon solvent of the oligomerization catalyst is any of benzene, toluene, a portion of fifth product stream, a portion of the fourth product stream, and a combination of the fifth and fourth product streams; and
the etheral solvent is one or more ethers.

13. The process of claim 1, wherein prior to step b-1):
the first stream of LAOs is quenched by admixture with a solvent capable of neutralizing dissolved catalyst species and removing them from the hydrocarbon phase of the first stream of LAOs, in which the solvent is a polar solvent comprising water, an alcohol, or a combination thereof; and
optionally the dissolved catalyst species are separated from the first stream of LAOs and recycled to the oligomerization reactor.

14. The process of claim 2, wherein the SPK comprises less than 1 ppm wt of any of Al, Ni, Cr, and combinations thereof.

15. The process of claim 2, wherein the SPK comprises greater than 65 wt % paraffins, and has a specific gravity in the range of 0.73-0.77 g/cm³ as measured by ASTM D287.

16. A SPK product prepared by a process according to claim 2, the SPK product comprising: greater than 65 wt % paraffins, a BI greater than 0.05, a specific gravity in the range of 0.73-0.77 g/cm³ as measured by ASTM D287, and a freezing point below −40° C., e.g. 47° C., as measured by ASTM D2386.

17. A plant for carrying out the process of claim 1, the plant comprising:
an oligomerization reactor arranged to receive: an ethylene stream and at least a portion of a second stream of C2-C6 olefins from a downstream first separation section; the oligomerization rector comprising an oligomerization catalyst, the oligomerization catalyst being a homogeneous catalyst comprising a transition metal compound or complex comprising at least one of Ti, Zr, Cr, Ni, together with an alkyl aluminum compound dissolved in a hydrocarbon solvent or an etheral solvent, or an alkyl aluminum compound dissolved in a hydrocarbon solvent or an etheral solvent, optionally as a co-catalyst; the oligomerization reactor further being arranged to provide a first stream of linear alfa olefins (LAOs) in the C2-C20 range of which at least 80 wt % having a Schultz-Flory distribution with a chain propagation probability (alpha) of from 0.35-0.60 or a Poisson distribution with a chain propagation probability (lambda) of from 1.0-4.7;
the first separation section arranged to receive the first stream of LAOs and to provide the second stream of C2-C6 olefins, and a third stream of C8+ olefins, the first separation section further comprising a conduit arranged for recycling the at least a portion of second stream of C2-C6 olefins to the oligomerization reactor;
optionally, a dewaxing reactor arranged to receive the third stream of C8+ olefins and hydrogen, the dewaxing reactor comprising a dewaxing catalyst, the dewaxing catalyst comprising at least one transition metal selected from Ni, Pd, Pt or combinations thereof, and optionally another transition metal, and a zeolite or zeotype or a silica-alumina; the dewaxing reactor further being arranged to provide a fourth product stream of branched C8-C20 isoparaffins; and
optionally, a second separation section arranged to receive the fourth product stream of branched C8-C20 isoparaffins and to provide a sixth stream of light end compounds and a fifth product stream as synthetic paraffinic kerosene (SPK), and wherein the SPK has i) a Branching Index, BI, defined as the average number of carbon atoms in the side chains divided by the average number of carbon atoms in the main chain, higher than 0.05, and ii) a freezing point below −40° C.

18. A method of retrofitting an existing hydroprocessing plant, comprising a dewaxing reactor and a separation section arranged downstream the dewaxing reactor; the method comprising:
installing an oligomerization reactor upstream of the dewaxing reactor, in which the oligomerization reactor is arranged to receive an ethylene stream and at least a portion of a second stream of C2-C6 olefins from a downstream first separation section; the oligomerization rector comprising an oligomerization catalyst, the oligomerization catalyst being a homogeneous catalyst comprising a transition metal compound or complex comprising at least one of Ti, Zr, Cr, Ni, together with an alkyl aluminum compound dissolved in a hydrocarbon solvent or an etheral solvent, or an alkyl aluminum compound dissolved in a hydrocarbon solvent or an etheral solvent, optionally as a co-catalyst; the oligomerization reactor further being arranged to provide a first stream of linear alfa olefins (LAOs) in the C2-C20 range of which at least 80 wt % having a Schultz-Flory distribution with a chain propagation probability (alpha) of from 0.35-0.60, or a Poisson distribution with a chain propagation probability (lambda) of from 1.0-4.7;
installing the first separation section downstream and in direct fluid communication with the oligomerization reactor, in which the first separation section is arranged to receive the first stream of LAOs and to provide the second stream of C2-C6 olefinsand a third stream of C8+ olefins;
installing a conduit in direct fluid communication between the first separation section and the oligomerization reactor, for recycling the at least a portion of the second stream of C2-C6 olefins from the first separation section to the oligomerization reactor;
installing a conduit in direct fluid communication between the first separation section and the dewaxing reactor, which is arranged downstream, for supplying the third stream of C8+ olefins to the dewaxing reactor, and in which the dewaxing reactor is further arranged to provide a fourth product stream of branched C8-C20 isoparaffins;
installing a conduit in direct fluid communication between the dewaxing reactor and the separation section arranged downstream of the dewaxing reactor for supplying the fourth product stream of branched C8-C20 isoparaffins to the separation section; the separation section is further arranged to provide a sixth stream of light end compounds and a fifth product stream as synthetic paraffinic kerosene (SPK), the SPK suitably having i) a Branching Index, BI, defined as the average number of carbon atoms in the side chains divided by the average number of carbon atoms in the main chain, higher than 0.05, and ii) a freezing point below −40° C.;
optionally, installing a hydrogen producing unit (HPU) or a gasification unit (GU) in direct or indirect fluid communication with the separation section arranged downstream the dewaxing reactor; the HPU being arranged to receive at least a portion of the sixth stream of light end compounds and provide a make-up hydrogen stream; and
installing a conduit for providing at least a portion of the make-up hydrogen gas to the dewaxing reactor.
